(19) United States
(12) Patent Application Publication
(10) Pub. No.: US 2016/0136103 A1
(43) Pub. Date: May 19, 2016

(54) Pharmaceutical Composition in the Form of Granules for the Treatment of Metabolic Disorders in Children

(71) Applicant: ASSISTANCE PUBLIQUE - HOPITAUX DE PARIS, PARIS Cedex 10 (FR)

(72) Inventors: VINCENT BOUDY, PARIS (FR); PASCALE DE LONLAY DEBENEY, VERSAILLES (FR); SANDRINE GRAFF DE FAGET, LA CELLE SAINT CLOUD (FR); MARIE-CAROLINE HUSSON OLLAGNIER, PARIS (FR)

(21) Appl. No.: 14/900,624

(22) PCT Filed: Jun. 26, 2014
(86) PCT No.: PCT/EP2014/063620

§ 371 Date: Dec. 22, 2015

(30) Foreign Application Priority Data
Jun. 27, 2013 (FR) 1356231

(51) Int. Cl. A61K 9/48; A61K 31/198; A61K 31/192

(52) U.S. Cl. CPC A61K 9/4866; A61K 31/192; A61K 31/198; A61K 9/4816; A61K 9/4833

(57) ABSTRACT

The present invention relates to a pharmaceutical composition for pediatric use and having controlled release, for treating metabolic disorders involving urea in children, said composition being in the form of granules behaving like a pseudofluid, said granules including a core consisting of sucrose, cellulose or isomalt particles, and of at least one active ingredient, said active ingredient having a water solubility of 200 g/L to 630 g/L of water, and at least one coating agent covering said core, the mean diameter of the microgranules being 0.1 mm to 1.2 mm and the granules having an angle of repose of less than 30.

### CROSS-REFERENCE TO RELATED APPLICATION

This application is the 35 U.S.C. §371 national stage application of PCT Application No. PCT/EP2014/063620, filed Jun. 6, 2014, which claims priority to and the benefit of, FR Patent Application No. 1356231, filed Jun. 27, 2013, both of which are herein incorporated by reference in their entirety.

### 1. FIELD OF THE INVENTION

The field of the invention is that of pharmaceutical formulations.

More specifically, the invention pertains to a pharmaceutical composition for pediatric use for the treatment of metabolic disorders, the composition being formulated in the form of granules enabling the controlled release of the active principle, the flow of these granules enabling easy production and distribution.

### 2. PRIOR ART

The urea cycle (see FIG. 1) is the metabolic means by which the nitrogen overload of the organism is eliminated. More specifically, ammonia is converted into urea by a succession of reactions that bring into play a total of six enzymes, including three mitochondrial enzymes (N-acetylglutamate synthetase NAGS; carbamoyl phosphate synthetase 1 CPS 1; ornithine transcarbamylase OTC) and three cytosolic enzymes (argininosuccinate synthetase ASS; argininosuccinate lyase ASL; arginase).

Disorders of the urea cycle can be of acquired origin, for example as a consequence of anti-epilepsy treatment with valproate. They are more commonly of hereditary origin: absence of synthesis, inadequate synthesis or synthesis in a non-functional form of one of the six enzymes of the urea cycle. However, whatever their cause, these disorders invariably lead to an accumulation of ammonium in the blood or hyperammonemia. The consequences of hyperammonemia are varied and can be serious: they include neurological disorders (mental retardation, encephalopathy, coma) and psychiatric disorders, liver damage (hepatic cytolysis, hepatic insufficiency, Reye's syndrome), possibly leading to the patient's death.

One of the main treatments used to limit the concentration of ammonium in blood is the administration of sodium benzoate (NaC₇H₅O₂), possibly associated with sodium phenylbutyrate. Sodium benzoate eliminates excess nitrogen by short-circuiting the urea cycle, through the use of latent metabolic pathways (acylation and acetylation systems: the acylation of glycine with benzoate produces hippuric acid which is excreted in the urine). The addition of phenylbutyrate also makes it possible, after acylation of glutamine, to obtain nitrogen excretion in the form of phenylacetylglutamine. The use of sodium benzoate enables the cleansing of one nitrogen atom (coming from glycine) per hippurate molecule excreted whereas the use of phenylbutyrate enables the cleansing of two nitrogen atoms (coming from glutamine) per phenylacetylglutamine molecule excreted.

At present, sodium benzoate is conditioned in the form of large capsules. Because of their volume, these capsules are difficult for children to swallow. To overcome this problem, pediatric healthcare teams as well as parents at home open the capsules and dissolve the sodium benzoate that they contain in water or fruit juice and give it to young patients to drink. However, since sodium benzoate is very bitter, children are generally recalcitrant about swallowing this solution.

In addition, the dosage of the treatment must naturally be adapted to the patient's weight and to the severity of his symptoms. More specifically, the dose administered to the patient depends on numerous factors such as weight, ammonemia and the severity of the pathological symptoms. Each patient therefore has a dosage proper to himself or to herself. Now, the dosage quantities in the capsules are standard and hence do not necessarily correspond to the dose that must be administered to every patient. Present-day treatment therefore is not compatible with the constraints of adapting dosages.

Besides, sodium benzoate is not the only treatment that the patients have to take. Indeed, it often happens that several medicines are associated in order to overcome the various deficiencies that patients may suffer.

Finally, the frequency of administration of each of these treatments varies between three and four times per day. The number of doses to be taken, which is fairly high for a child, makes the treatment painful and tiresome. The patient's pace of living then revolves around the times of administration of his medicine. This inevitably causes frustration among children especially when the treatment has to be taken for life.

Another aspect is that few schools allow treatment to be administered in their premises, for obvious reasons of safety and responsibility. Therefore, frequent administrations of sodium benzoate or any other therapeutic molecule make it difficult for the child to attend school.

For all these reasons, it is very difficult to obtain efficient compliance with treatment among young patients.

The invention is aimed especially at overcoming these drawbacks of the prior art.

More specifically, it is the goal of the invention, in at least one embodiment, to provide a pharmaceutical composition for the treatment of metabolic illnesses in children that is easy to swallow.

It is another goal of the invention, in at least one embodiment, to propose a pharmaceutical composition which makes it possible to reduce the frequency of administrations of the medicine.

It is yet another goal of the invention, in at least one embodiment, to provide a pharmaceutical composition whose dosage can easily be adapted by the healthcare personnel, the patient or his carers.

It is also a goal of the invention, in at least one embodiment, to provide a pharmaceutical composition that enables efficient compliance with treatment by the patients of pediatric departments.

### 3. SUMMARY OF THE INVENTION

These goals as well as others that shall appear here below are achieved by means of a pharmaceutical composition for pediatric use and with controlled release, for the treatment of metabolic urea disorders in children.

According to the invention, such a composition takes the form of granules which behave as a pseudo-fluid, said granules comprising a core constituted by a particle of saccharose, cellulose or isomalt and at least one active principle, said active principle having solubility in water of 200 g/L to 630 g/L of water, and at least one coating agent covering said core, the mean diameter of the granules ranging from 0.2 mm to 1.2 mm and the granules having an angle of repose of less than 30°.

The term “angle of repose” is understood to be the angle formed by a molecule of granular material with a horizontal plane. More specifically, when a cone is formed with a granular material, there is a critical value of an angle between the slope of the cone and the horizontal plane beyond which the cone collapses. The formula for calculating the angle of repose is as follows: α=arctan (2 h/d) in which

α is the value of the angle of repose,

h is the height of the cone of granular material, and

d is the diameter of the base of this same cone.

The solubility of the active principle is determined in g/L of pure water, at a temperature of 37° C.±0.5° C.

The term “control of the release of the active principle” is understood to mean the capacity to modulate the release of the active principle by choosing immediate, extended or delayed release. For example, the choice of coating makes it possible to decide which part of the organism will have active principle released in it.

Thus, the invention relies on a wholly novel and original approach proposing a novel galenic formulation enabling improved compliance with the treatment.

A first advantage of the composition according to the invention is that it can be swallowed directly by the patient. It is no longer necessary to dissolve the active principle in powder, and this considerably facilitates the work of nurses and the compounders in hospital pharmacies. This characteristic also limits losses of product at the bottom and on the walls of the glass. Consequently, the dose effectively absorbed by the patient is closer to the dose administered.

A second advantage is that this formulation makes it possible to adapt the treatment dose more easily to the patient. In other words, the weight of a child is highly variable at the growth stage, and the dose of the treatment therefore needs to vary according to this weight. In addition, certain patients can have more severe forms of illness than others. The composition according to the invention makes it possible to easily adapt the dosage of the treatment to the patient in taking only the exact dose. For example, the composition of the invention can be packaged in a dosing syringe. It is also easy to take the right dose by simply actuating the dosing piston of the syringe. The composition can also be packaged in bulk and dosed by means of a dosing spoon. In this case, it is enough simply to take out the accurate number of spoonfuls.

Another advantage of the composition in granular form is that it behaves like a pseudo-fluid. The term “pseudo-fluid” is defined as the flow of a granular material similar to that of a fluid. In other words, the quality of this flow is excellent and this facilitates the manufacture of the composition according to the invention. In addition, the granules according to the invention are spherical, thus enabling a reproducible stacking of the granules. This reproducibility of the granules makes it possible, for a given recipient, on the one hand to reproduce the dose from one administration to the next and secondly to easily measure the dose to be administered to the patient.

The use of sugar particles is particularly interesting when the treatment of these enzyme disorders requires the associated intake of saccharose.

Besides, the results in terms of the kinetics of dissolution or rate of dissolution have shown differences depending on the nature of the particle.

Advantageously, the degree of sphericity of the granules is at least 90%. The sphericity of the granules is great in order to achieve the goal of quality of flow. In a particularly advantageous way, the degree of sphericity of the granules is at least 95%.

The sphericity of the granules is measured by any method well known to those skilled in the art. In particular, the sphericity S of the granules is determined by the following formula: S=D1/D2 in which D1 is the minimum measured diameter of a granule and D2 is the maximum measured diameter of a granule.

Coating furthermore masks the bitter and unpleasant taste of sodium benzoate. It is thus more pleasant for a patient, and all the more so for a child, to swallow his dose and contribute to good compliance. In this respect, it is possible to envisage adding aromatic flavors to the preparation in order to improve the flavor of the composition according to any technique well known to those skilled in the art.

The coating also protects the active principle from deterioration by protecting it from moisture, UV and other interactions with the environment. As a consequence, coating makes it possible to control the duration of conservation of the composition. Another advantage of the composition of the invention is that it also enables the controlled release of the active principle, and especially of sodium benzoate in the patient's organism. It is possible, with the composition of the invention, to reduce the administrations from four or five administrations per day with the prior-art compositions to only one to two administrations per day with the composition of the invention, in doing so by enabling immediate release, extended release or delayed release. Since the number of administrations is reduced, the tiresome character of the treatment is lowered. The risk related to forgetting to take medicine during the day is reduced, since the treatment takes up a smaller part of the patient's life and the patient's relationship with his treatment is thereby equivalently improved. In addition, this reduction of the number of administrations enables a child to go to school. This characteristic therefore improves the child's compliance with the treatment. The term “child” or “young patient” or “pediatric patient” is understood to mean an individual whose age ranges from six months to 11 years.

It is in addition possible, as understood in the invention, to envisage the formulation of a composition by combining several active principles so as to reduce the number of administrations and medicines to be taken for a same patient.

In one variant of the invention, the granules of the composition according to the invention can comprise several layers: a core particle in which a first active principle is powdered and then a first coating above which a second active principle is deposited before a second and last coating. It is possible according to the invention to formulate granules comprising several layers of active principles separated by a coating layer. The core particle is chosen from among the particles of saccharose, isomalt or cellulose.

The coating also protects handlers from contact with the active principles. Thus, the coating also prevents interaction between the different active principles and the different chemical agents used in the preparation of the composition according to the invention.

Preferably, the active principle is chosen from among sodium benzoate, citrulline, glycine, mannose, L-carnitine or one of their pharmaceutically acceptable salts. In a first variant of the invention, the composition comprises 10% to 75% by weight of sodium benzoate or one of its pharmaceutically acceptable salts, and 0.1% to 20% by weight of coating agent.

This variant makes it possible especially to efficiently mask the bitter taste of sodium benzoate while enabling controlled release of sodium benzoate in the patient's organism for several hours with the effect of delay or extension of the release for several hours.

In another advantageous variant of the invention, the composition comprises 10% to 75% by weight of citrulline or one of its pharmaceutically acceptable salts, and 0.1% to 20% by weight of coating agent.

Citrulline acts as a hypo-ammonemia agent. It has indeed been shown to be efficacious in cases of X-linked transmission of OTC deficiency or again in the treatment of H syndrome (impaired mitochondrial transport related to chromosome 13) and intolerance to dibasic proteins. Its hypo-ammonemia properties are also the basis of its use as an anti-asthenic in the treatment of muscular fatigue by citrulline malate.

In one equally advantageous variant, the composition according to the invention comprises 10% to 75% by weight of glycine or one of its pharmaceutically acceptable salts and 0.1% to 20% by weight of coating agent.

In another advantageous variant of the invention, the composition comprises 10% to 75% by weight of mannose or one of its pharmaceutically acceptable salts and 0.1% to 20% by weight of coating agent.

In another advantageous variant of the invention, the composition comprises 10% to 75% by weight of L-carnitine or one of its pharmaceutically acceptable salts and 0.1% to 20% by weight of coating agent.

Preferably, said coating agent is a polymer chosen from among polyvinyl acetate, the methacrylate copolymers and ethyl cellulose. In an even more preferred way, the coating agent is polyvinyl acetate.

The inventors have noted that the particular choice of these agents makes it possible firstly to efficaciously mask the bitter flavor of the active principles, and secondly to control the release of the active principle that they contain.

Advantageously, the active principle is released in an extended manner, at physiological pH, for a duration of 3 hours to 8 hours, preferably for 4 to 7 hours, even more preferably for 5 hours.

Advantageously, the active principle is released in a delayed manner, at physiological pH, for a duration of 30 minutes to 2 hours.

Advantageously, the active principle is released rapidly, at physiological pH, for the first 2 hours with masking of the flavor in order to improve compliance with the treatment.

Preferably, the core is constituted by saccharose, cellulose or isomalt particles. In an even more preferred manner, the core is constituted by saccharose particles because this type of core makes it possible to obtain more spherical particles.

The nucleus or core makes it possible to have a first surface on which the solution of active principle is sprayed during the manufacture of the granules. The sugar, cellulose or isomalt particles all equally well enable adequate adhesion of the solution and adequate re-release of the active principle over time. Examples of cores that can be cited are those commercially available under the references GalenIQ® (Colorcon UK, or Beneo-Palatinit GmbH, Germany) and Suglets® (Colorcon UK). Cellulose particles suited to implementing the invention are commercially distributed under the reference Cellets® (Pharmatrans Sanaq AG, Germany).

Advantageously, the composition according to the invention furthermore comprises a plasticizing agent and/or a lubricating agent.

These additives facilitate the manufacture of the granules. Examples of plasticizing agents that can be cited are triethyl acetate or propylene glycol. An example of lubricating agent that can be cited is micronized talc. These are low-cost constituents and further facilitate the method for manufacturing the composition according to the invention.

Another object of the invention is a method for manufacturing the composition according to the invention comprising the following steps:
spraying a solution of active principle on particles of saccharose, cellulose or isomalt, the active principle being chosen from among sodium benzoate, citrulline, glycine, mannose, L-carnitine or one of their pharmaceutically acceptable salts, at a temperature of 40-60° C. and a pressure of 2-4 bars,
drying at a temperature of 40-70° C. for 5-20 minutes,
spraying a solution comprising at least one coating agent and/or one plasticizing agent and/or at least one lubricating agent at a temperature of 25-40° C. and a pressure of 1-2 bars;
drying the micro-granules for 2-20 hours at 60° C. This step is also called “curing”.

Yet another goal of the invention is a method for treating urea cycle disorders in patients aged six months to 11 years.

More specifically, a method of treatment of this kind comprises the administration, once to three times per day, of a composition with extended, immediate or delayed release comprising granules, the core of which comprises an active principle chosen from among sodium benzoate, citrulline and glycine or one of their pharmaceutically acceptable salts.

### 4. LIST OF FIGURES

Other features and advantages of the invention shall appear more clearly from the following description of a preferred embodiment, given by way of a simple, illustratory and non-exhaustive example and from the appended figures, of which:

FIG. 1 is a diagram representing the metabolic urea cycle;

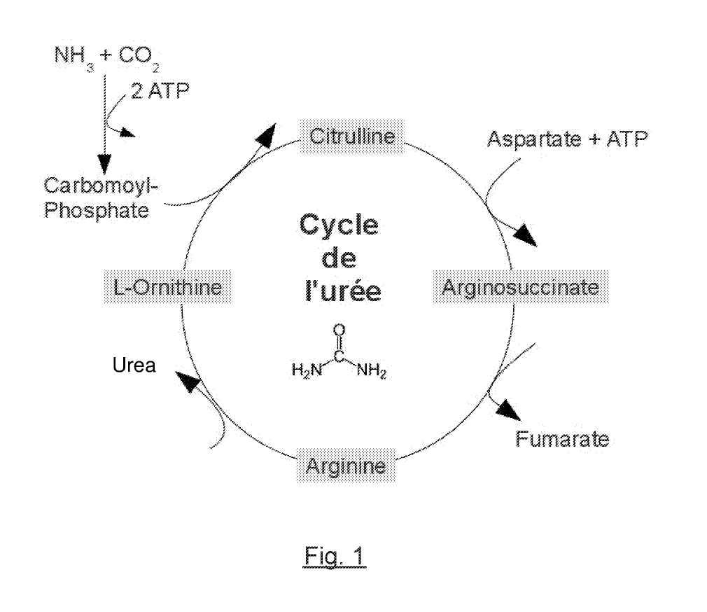

FIG. 2 is a graph representing the kinetics of release of sodium benzoate as an active principle, formulated in the form of granules according to the invention, with different combinations of coating agents or without coating, with isomalt core particles (commercially distributed as GalenIQ 980);

FIG. 3 is a graph representing the kinetics of release of sodium benzoate as an active principle, formulated in the form of granules according to the invention, with different combinations of coating agents or without coating, with saccharose core particles (commercially distributed by the name of Suglets 8/20);

FIG. 4 is a graph representing the kinetics of release of sodium benzoate, formulated in the form of granules according to the invention, with a saccharose core, in solutions at pH 6.8 and pH 1.

### 5. DESCRIPTION OF ONE EMBODIMENT OF THE INVENTION

The general principle of the invention relies on the novel formulation of active principles intended for treating the consequences and/or the symptoms of urea cycle disorders in order to improve compliance by children with the treatment. More specifically, the composition of the invention masks the taste of the active principle through the coating which makes it more pleasant for the patient. The composition according to the invention also enables control over the duration of release of the active principle into the patient's organism. This control over the release of the active principle into the patient's organism improves the efficacy of the treatment. Thus, the number of administrations per day is reduced, the risk of forgetting to take a dose is restricted and the patient's lifestyle is no longer subjected to the pace of his treatment. In addition, the size and the spherical shape of the granules enables the composition of the invention to behave like a pseudo-fluid facilitating firstly the manufacture of the composition and secondly its distribution. The granules also make it possible to adapt the patient's dosage more easily to his weight and the severity of his illness. All these characteristics improve compliance with the treatment.

5.1. Preparation of Sodium Benzoate Granules

According to the invention, a composition of sodium benzoate is prepared in granular form by means of a fluidized bed.

Sodium benzoate in powder form is first of all diluted in pure water in a concentration of 450 g/L. The solution thus obtained is sprayed on isomalt core particles commercially distributed under the reference GalenIQ® 980 (Beneo-Palatinit GmbH, Germany) or saccharose commercially distributed under the reference Suglets 18/20® (Colrcon, Draft, United Kingdom). The size of these isomalt particles ranges from 700 to 1000 μm and the size of the saccharose particles ranges from 850 to 1000 μm. The temperature of the sprayed sodium benzoate solution is in the 45-50° C. range and the pressure is in the 3-4 bar range. The diameter of the spraying nozzle is about 0.5 mm.

The particles of sodium benzoate thus obtained are then dried in hot air at 50-60° C. for about five minutes.

The coating solution is then sprayed at a temperature of 25-40° C. and a pressure of 1-2 bars. The coating solution comprises a coating polymer and optionally a plasticizing agent and/or a lubricating agent. The plasticizing agent gives a smooth and bright appearance to the granules while the talc lubricates the surface of the granules in order to limit friction between them.

The granules thus coated are then dried in an oven for two hours, at a temperature of 60° C.

Three different coating agents were tested:
polyvinyl acetate commercially distributed as Kollicoat® SR30D, and
ethyl cellulose commercially distributed as Aquacoat® ECD30, and
a copolymer of methacrylate commercially distributed as Eudragit® RS 30D.

The different compositions of the coating solutions are indicated here below. The quantities are given in percentage by mass for the composition of dry coating non-diluted in water:

TABLE 1
Compositions of the coating solutions

| | Coating | | |
|---|---|---|---|
| | solution 1 | Coating solution 2 | Coating solution 3 |
| Coating | Kollicoat ® | Aquacoat ® ECD30 | Eudragit ® RS 30D |
| agent | SR30D QSP | QSP 100% | QSP 100% |
| | 100% | | |
| Plasticizing | Propylene | Triethyl acetate 24% | Triethyl acetate 10% |
| agent | glycol 10% | | |
| Lubricating | Micronized | — | Micronized talc |
| agent | talc 33% | | |

On an average, the granules comprise about 50% by weight of sodium benzoate and 50% by weight of coating agent. The sugar particle used as a support for the spraying of the active agent and of the coating agent was not taken into account in this calculation of the weight ratio.

5.2 Evaluation of the Physical Characteristics of the Granules

The size and shape of the granules are studied and measured under the microscope. The granules are photographed under the microscope. The photographs are then digitized and processed by the computer program Videomet. The sphericity S of the granules is determined by the following formula: S=D1/D2 in which D1 is the minimum measured diameter of a granule and D2 is the maximum measured diameter of a granule. The sphericity of the different batches ranges from 95%-97%. The average size of the granules is about 1 mm.

The friability of the granules is also measured as follows: the granules are weighed and then introduced into a Eur. Ph. Friabilitor type drum with glass beads having a diameter of 1 mm. The drum is made to rotate at a speed of 25 rotations per minute for ten minutes. The percentage of friability corresponds to the loss of weight of the particles at the end of the test related to its initial weight. Whatever the batch, the percentage of friability is less than 1%. The method of the invention therefore produces shock-resistant granules.

The Hausner ratio was also measured in order to evaluate the fluidity of the granules. The granules of each batch were placed in a graduated 200 ml cylinder. The cylinder was then tapped 1250 times in order to settle the granules. The difference between the volume before and after the test makes it possible to determine the fluidity of the granules. For each batch, the Hausner ratio ranges from 1.01 to 1.02. This result shows that there are few frictional forces between the particles for which the flow is fluid enough to reach equilibrium without being settled.

The angle of repose is also measured for 100 g of granules of each batch. The granules are introduced into a funnel made of stainless steel with a height of 13.6 cm, a greater diameter of 11 cm and a smaller diameter of 6 cm. The funnel is placed at 7.8 cm above a horizontal plane. The granules form a cone, the dimensions of which have been measured by laser. The angle of repose is calculated according to the following formula: α=arctan (2 h/d)

where α is the value of the angle of repose,

h is the height of the cone of granular material, and

d is the diameter of the base of this cone.

The results indicate an angle of repose ranging from 29° to 30°. An angle of repose below or equal to 30° indicates excellent fluidity of the composition.

In conclusion, given the results obtained, the composition according to the invention, whatever the batch, behaves like a pseudo-fluid. There is little friction between the particles. The flow can be compared to that of a fluid like water.

5.3 Evaluation of the Chemical Characteristics of the Granules

Granules of each batch were incubated in pure water in order to measure their time for releasing the active principle.

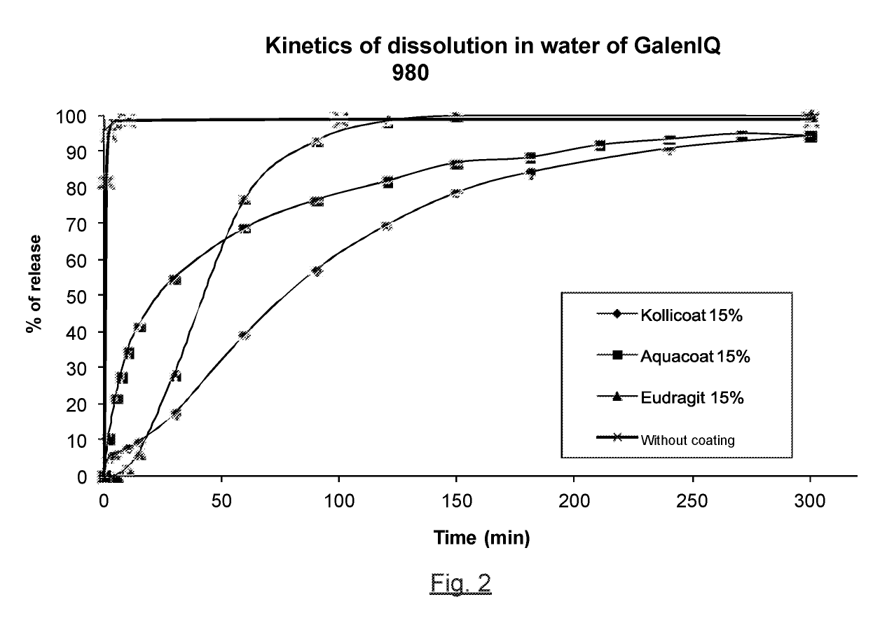

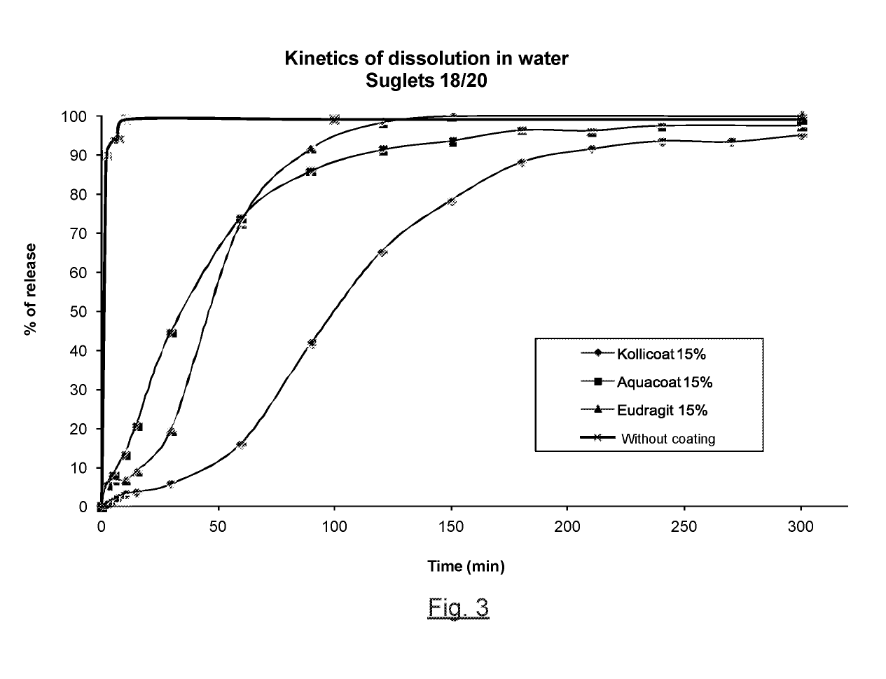

FIGS. 2 and 3 present the results obtained with each of the compositions manufactured in taking account of the initial core particle and of the coating composition used. The kinetics of dissolving were also measured for the particles alone associated with sodium benzoate without coating, as a negative control for the experiment.

As can be observed, the nature of the core particle made of sugar or isomalt had little influence on the speed of release of the active principle in water. The sugar particles (FIG. 3) however enable slower release of the active principle as compared with the isomalt particles (FIG. 2).

Coating with a polyvinyl acetate (Kollicoat® SR30D) enables a slower release than with ethyl cellulose and the polymethacrylate copolymer. Besides, the granules coated with polyvinyl acetate or polymethacrylate copolymer begin to fragment only after about ten minutes, which is a time sufficient to enable swallowing and to prevent the taste of the active principle from being felt by the patient's taste buds.

The kinetics of release of the granules in a medium close to that of the digestive tube were also evaluated by incubating the granules in a solution of hydrochloric acid with a pH 1 or pH 6.8 for two hours. This experience was intended to simulate the release of active principle and the breaking up of the granules in an environment chemically similar to that of the gastric juices and then of the intestinal tract.

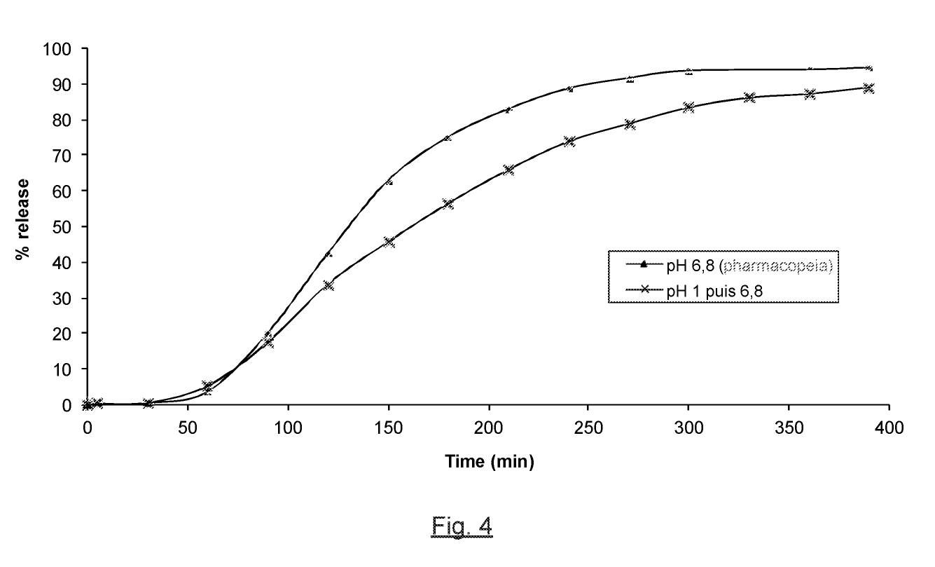

The results obtained with the batch of sodium benzoate granules, the coating agent of which is polyvinyl acetate (Kollicoat® SR30D) associated with propylene glycol as a plasticizing agent (coating solution 1) are presented in FIG. 4. The experiment was conducted three times, and the present results indicate the average value. The curve with triangular patterns and solid lines indicate the results obtained in a hydrochloric acid solution at pH 6.8. The curve with cross-like patterns indicates the results obtained at pH 1 for one hour and then at pH 6.8 for the rest of the dissolution trial.

As can be seen in FIGS. 2 and 3, the release of the active principle is slower during tests involving a first phase at pH 1 and then a second phase at pH 6.8. The granules show high resistance to the release of the active principle in the stomach.

The maximum amount of active principle is released at the end of eight hours. About 80% of the active principle is released at the end of 3 h20 minutes.

### 6. CONCLUSION

The composition of the invention enables the formulation of active principles in the form of granules used firstly to mask their flavor and secondly to control the kinetics of release in the patient's organism in order to reduce the number of dose administrations.

In addition, the composition in the form of granules behaves like a pseudo-fluid. This enables optimum flow of the composition.

The composition according to the invention can also be packaged in bulk or in a dosing device which adapts the dosage of the composition more easily according to the progress of the patient's weight.

1. Pharmaceutical composition for pediatric use and with controlled release for the treatment of metabolic urea disorders in children, said composition taking the form of granules behaving like a pseudo-fluid, said granules comprising a core constituted by a particle of saccharose, cellulose or isomalt and at least one active principle, said active principle having solubility in water of 200 g/L to 630 g/L of water, and at least one coating agent covering said core, the mean diameter of the granules ranging from 0.2 mm to 1.2 mm and the granules having an angle of repose of less than 30°.

2. Composition according to claim 1, wherein the active principle is chosen from among sodium benzoate, citrulline, glycine, mannose, L-carnitine or one of their pharmaceutically acceptable salts.

3. Pharmaceutical composition according to claim 2, comprising 10% to 75% by weight of sodium benzoate or one of its pharmaceutically acceptable salts, and 0.1% to 20% by weight of coating agent.

4. Pharmaceutical composition according to claim 2, comprising 10% to 75% by weight of citrulline or one of its pharmaceutically acceptable salts, and 0.1% to 20% by weight of coating agent.

5. Pharmaceutical composition according to claim 2, comprising 10% to 75% by weight of glycine or one of its pharmaceutically acceptable salts and 0.1% to 20% by weight of coating agent.

6. Pharmaceutical composition according to claim 1, wherein said coating agent is a polymer chosen from among polyvinyl acetate, the methacrylate copolymers and ethyl cellulose, preferably polyvinyl acetate.

7. Composition according to claim 1, wherein the active principle is released at physiological pH for a duration of 3 hours to 8 hours, preferably 5 hours.

8. Pharmaceutical composition according to claim 1, wherein said core is made of saccharose.

9. Composition according to claim 1, further comprising a plasticizing agent and/or a lubricating agent.

10. Composition according to claim 1, wherein the degree of sphericity of the granules is at least 90%, preferably at least 95%.

11. Method for manufacturing the composition according to claim 1, comprising the following steps:
spraying a solution of active principle on particles of saccharose, cellulose or isomalt, the active principle being chosen from among sodium benzoate, citrulline, glycine, mannose, L-carnitine or one of their pharmaceutically acceptable salts, at a temperature of 40-60° C. and a pressure of 2-4 bars,
drying at a temperature of 40-70° C. for 5-20 minutes,
spraying a solution comprising at least one coating agent and/or one plasticizing agent and/or at least one lubricating agent at a temperature of 25-40° C. and a pressure of 1-2 bars;
drying the micro-granules for 2-20 hours at 60°.
